(19) United States
(12) Patent Application Publication
(10) Pub. No.: US 2014/0079781 A1
(43) Pub. Date: Mar. 20, 2014

(54) PHARMACEUTICAL COMPOSITIONS

(71) Applicants: Peter SCHNEIDER, Ulm-Einsingen (DE); Thorsten NEUHAUS, Biberach an der Riss (DE)

(72) Inventors: Peter SCHNEIDER, Ulm-Einsingen (DE); Thorsten NEUHAUS, Biberach an der Riss (DE)

(73) Assignee: BOEHRINGER INGELHEIM INTERNATIONAL GMBH, Ingelheim am Rhein (DE)

(21) Appl. No.: 14/087,388

(22) Filed: Nov. 22, 2013

Related U.S. Application Data
(63) Continuation of application No. 13/100,328, filed on May 4, 2011.

(30) Foreign Application Priority Data
May 5, 2010 (EP) 10162067.2
Apr. 28, 2011 (EP) 11164203.9

(51) Int. Cl. A61K 9/28; A61K 31/4439; A61K 31/522

(52) U.S. Cl. CPC A61K 9/2866; A61K 31/522; A61K 31/4439; A61K 9/282; A61K 9/2813
USPC 424/480; 514/263.21; 424/474

(57) ABSTRACT

The present invention relates to pharmaceutical compositions comprising fixed dose combinations of a DPP-4 inhibitor drug and pioglitazone, processes for the preparation thereof, and their use to treat certain diseases.

The present invention relates to pharmaceutical compositions comprising a fixed dose combination (FDC) of a DPP-4 inhibitor drug and pioglitazone (particularly pioglitazone hydrochloride), processes for the preparation thereof, and their use to treat certain diseases.

In a more detailed aspect, the present invention relates to a pharmaceutical composition, particularly a solid preparation (e.g. an oral solid dosage form) of a selected dipeptidyl peptidase-4 (DPP-4) inhibitor (particularly linagliptin) and pioglitazone (particularly pioglitazone hydrochloride).

In a further more detailed aspect, the present invention relates to a pharmaceutical composition, particularly a solid preparation (e.g. an oral solid dosage form, such as e.g. a tablet, particularly for immediate drug release), comprising

a first composition comprising pioglitazone (particularly pioglitazone hydrochloride) and one or more excipients, and
a second composition comprising a selected dipeptidyl peptidase-4 (DPP-4) inhibitor (particularly linagliptin) and one or more excipients.

In a yet further more detailed aspect, the present invention relates to a pharmaceutical composition, particularly a solid preparation (e.g. an oral solid dosage form, such as e.g. a tablet, particularly for immediate drug release), comprising the following first and second components or parts:

the first component or part comprising pioglitazone (particularly pioglitazone hydrochloride) and one or more excipients,
the second component or part comprising a selected dipeptidyl peptidase-4 (DPP-4) inhibitor (particularly linagliptin) and one or more excipients.

An aim of the present invention is to provide a pharmaceutical composition comprising a combination of a selected DPP-4 inhibitor (particularly linagliptin) and pioglitazone (particularly pioglitazone hydrochloride).

A further aim of the present invention is to provide a pharmaceutical composition comprising a selected DPP-4 inhibitor (particularly linagliptin) and/or pioglitazone hydrochloride by which undesired interactions or incompatibilities between any components, e.g. incompatibilities of any of the active ingredients with certain excipients (which may result in significant degradation of one or both active ingredients and/or which may result in inadequate chemical and/or physical stability of the composition, such as e.g. time-course decomposition of active ingredients, decreased activity, degraded storage or dissolution stability such as time-course changes in the active ingredient dissolution) can be overcome.

A further aim of the present invention is to provide a pharmaceutical composition comprising a selected DPP-4 inhibitor (particularly linagliptin) and pioglitazone hydrochloride by which incompatibilities of the active ingredients with one another (which may result in significant degradation of one or both of the active ingredients and/or which may result in inadequate chemical and/or physical stability of the composition, such as e.g. time-course decomposition of active ingredients, decreased activity, degraded storage or dissolution stability such as time-course changes in the active ingredient dissolution) can be overcome.

A further aim of the present invention is to provide a pharmaceutical composition comprising linagliptin and pioglitazone hydrochloride which shows no signs or only marginal signs of change, incompatibility or degradation of linagliptin and/or pioglitazone hydrochloride and thus enables a sufficient physical and/or chemical stability, shelf life and/or dissolution profile.

A further aim of the invention is to provide a pharmaceutical composition comprising linagliptin and pioglitazone hydrochloride which has high content uniformity and/or which allows an effective production with regard to time and costs of pharmaceutical dosage forms.

A further aim of the invention is to provide a pharmaceutical dosage form (particularly for oral administration) comprising linagliptin and pioglitazone hydrochloride which has good chemical and/or physical stability, which has a good shelf life, which has a short disintegration time, which has good dissolution properties and/or which enables a high bioavailability of the active ingredients in a patient.

A further aim of the invention is to provide a pharmaceutical dosage form (particularly for oral administration) comprising linagliptin and pioglitazone hydrochloride which is sufficiently (chemically and/or physically) stable, which displays similarity of immediate drug release and/or of in-vitro dissolution profiles and/or is bioequivalent to the free combination and/or which maintains the original dissolution profiles of corresponding mono tablets of each of the individual entity drug products (linagliptin and pioglitazone (e.g. Actos) or pioglitazone mono or combination market tablets).

Further aims of the present invention become apparent to the one skilled in the art by the description hereinbefore and in the following (including the examples).

The enzyme DPP-4 also known as CD26 is a serine protease known to lead to the cleavage of a dipeptide from the N-terminal end of a number of proteins having at their N-terminal end a prolin or alanin residue. Due to this property DPP-4 inhibitors interfere with the plasma level of bioactive peptides including the peptide GLP-1 and are considered to be promising drugs for the improvement of glycemic control and for the treatment of diabetes mellitus, particularly in type 2 diabetes patients.

For example, DPP-4 inhibitors and their uses, particularly their uses in metabolic (especially diabetic) diseases, are disclosed in WO 2002/068420, WO 2004/018467, WO 2004/018468, WO 2004/018469, WO 2004/041820, WO 2004/046148, WO 2005/051950, WO 2005/082906, WO 2005/063750, WO 2005/085246, WO 2006/027204, WO 2006/029769 or WO2007/014886; or in WO 2004/050658, WO 2004/111051, WO 2005/058901 or WO 2005/097798; or in WO 2006/068163, WO 2007/071738 or WO 2008/017670; or in WO 2007/128721, WO 2007/128724 or WO 2007/128761, or WO 2009/121945.

A DPP-4 inhibitor within the meaning of the present invention includes, without being limited to, any of those DPP-4 inhibitors mentioned hereinabove and hereinbelow, preferably orally active DPP-4 inhibitors.

In a closer embodiment, a DPP-4 inhibitor within the meaning of the present invention includes a DPP-4 inhibitor with an amino group, especially a free or primary amino group.

In a yet closer embodiment, a DPP-4 inhibitor in the context of the present invention is a DPP-4 inhibitor with a primary amino group, particularly with a free primary amino group.

In a particularly preferred embodiment of this invention the DPP-4 inhibitor is linagliptin (also named BI 1356).

In attempts to prepare pharmaceutical compositions of selected DPP-4 inhibitors it has been observed, that the DPP-4 inhibitors with a primary or secondary amino group show incompatibilities, degradation problems, or extraction problems with a number of customary excipients such as microcrystalline cellulose, sodium starch glycolate, croscarmellose sodium, tartaric acid, citric acid, glucose, fructose, saccharose, lactose, maltodextrines, polyethylene glycol 400. Though the compounds themselves are very stable, they react with incompatible partner drug, or its impurity product, and/or with many excipients used in solid dosage forms and with impurities of excipients, especially in tight contact provided in tablets and at high excipient/drug ratios. The amino group appears to react with reducing sugars and with other reactive carbonyl groups and with carboxylic acid functional groups formed for example at the surface of microcrystalline cellulose by oxidation. These difficulties may be primarily observed in low dosage ranges of the DPP-4 inhibitor used, which are required due to their surprising potency, and/or high dosage ranges of the partner drug used.

Further, the DPP-4 inhibitors which have a primary or secondary amino group may show incompatibilities with pioglitazone hydrochloride (which may act as proton donator to the amino group), especially in tight contact provided in tablets and/or in the presence of water and/or under application of compaction forces. The incompatibilities of these DPP-4 inhibitors with pioglitazone hydrochloride can lead to chemical instability, disproportionation of pioglitazone hydrochloride and/or degradation of the DPP-4 inhibitors in the presence of pioglitazone hydrochloride with the consequence of compromised physical stability of the composition.

One stabilizing principle for such compositions may be the use of a stabilizer such as L-arginine. However, prototype tablets which comprise linagliptin, pioglitazone hydrochloride and L-arginine as stabilizer show indeed good (chemical) stability against drug degradation, but at higher moisture conditions (e.g. r.h. >62%) such tablets show physical instability and tablet core damages, presumably due to interaction with the excipients.

Further, pioglitazone hydrochloride is practically insoluble in water. Particularly, pioglitazone hydrochloride shows very poor solubility in weak acidic and neutral to basic media while it shows slightly better solubility in strongly acidic media. For pioglitazone hydrochloride the intrinsic dissolution rates in aqueous media are only above 1000 μg/cm²/min at pH 1, whereas for less acidic solutions (e.g. pH 2) the intrinsic dissolution rates are below 100 μg/cm²/min. Therefore, the intrinsic dissolution rate of pioglitazone hydrochloride may be rate limiting for dissolution/absorption of the composition and pose an additional risk for providing similar dissolution profiles of pioglitazone hydrochloride in the composition to the original mono tablets (e.g. Actos) or combination market tablets (e.g. Duetact, Competact) and/or for matching bioequivalence with the original mono or combination market tablets.

Further, it is another goal to choose, if possible, the excipients for use together with the DPP-4 inhibitor (particularly linagliptin) possibly similar to those for use together with pioglitazone hydrochloride, e.g. in order to minimize stability risks and/or to optimize adhesion of layers or components, if the active ingredients are present in different layers or components.

Therefore, pharmaceutical compositions are required to overcome and solve these technical problems.

It has now been found that the pharmaceutical compositions, formulations, preparations and dosage forms which are described in greater details herein, have surprising and particularly advantageous properties, which make them particularly suitable for the purposes and aims of this invention.

Thus, the present invention relates to a pharmaceutical composition comprising or made of

a) a first composition, ingredient, component or part comprising or made of pioglitazone or a pharmaceutically acceptable salt thereof, and, optionally, one or more excipients;
b) a second composition, ingredient, component or part comprising or made of a DPP-4 inhibitor or a pharmaceutically acceptable salt thereof, and, optionally, one or more excipients;
and, optionally, one or more excipients.

In one aspect, it has been found that by individually preparing the first part (composition) containing pioglitazone hydrochloride and one or more excipients, and the second part (composition) containing the DPP-4 inhibitor (particularly linagliptin) and one or more excipients, and forming a composition (solid preparation) containing these two parts, an adverse influence (e.g. degradation, inadequate chemical and/or physical stability, such as e.g. initial or time-course decomposition of active ingredients, decreased activity, degraded storage or dissolution stability such as time-course changes in the active ingredient dissolution) caused by the interaction of the active ingredients with one another and/or with certain excipients of the other part can be suppressed and the dissolution rate of each active ingredient can be optimized.

Preferably, within the compositions according to this invention, pioglitazone hydrochloride and the DPP-4 inhibitor (particularly linagliptin) are separated (preferably physically separated) from each other and/or the contact area of the two portions is reduced or minimized, such as e.g. in form of a bilayer tablet (for example, wherein the first layer comprises the first portion and the second layer comprises the second portion).

The present invention further relates to a pharmaceutical composition comprising:
(1) a first part or composition comprising pioglitazone or a pharmaceutically acceptable salt thereof, and one or more excipients;
(2) a second part or composition comprising a DPP-4 inhibitor or a pharmaceutically acceptable salt thereof, and one or more excipients.

The present invention further relates to a pharmaceutical composition, particularly for oral administration, comprising the following first and second parts:
(1) the first part comprising pioglitazone or a pharmaceutically acceptable salt thereof, and one or more excipients;
(2) the second part comprising a DPP-4 inhibitor, particularly linagliptin, or a pharmaceutically acceptable salt thereof, and one or more excipients.

Particularly, the present invention relates to a pharmaceutical composition (e.g. solid preparation or solid oral dosage form, e.g. tablet, particularly for immediate release) comprising the following first and second parts:
(1) the first part comprising or made of pioglitazone hydrochloride, and one or more excipients;
(2) the second part comprising or made of linagliptin, and one or more excipients.

In general, excipients which may be used may typically be selected from the group consisting of one or more diluents or fillers, one or more binders, one or more disintegrants, one or more lubricants, and the like.

Optionally, excipients which may be used may comprise one or more further additives conventionally used in the field of pharmaceutical preparation, such as e.g. excipients other than those described before, for example colorants, pH adjusting agents, stabilizers, surfactants, flavors, glidants, coating bases and/or coating additives and the like.

Preferably the excipients used are pharmaceutically acceptable and may be selected from those conventionally employed in the field of pharmaceutical preparation. In the following the excipients and carriers in the pharmaceutical compositions, formulations, preparations, parts and dosage forms of this invention are described in further detail.

The first and second parts in the solid composition of the present invention mean compositions or constitution components, which each may be capable of existing as an independent composition. Thus, each part may be an individual aspect of the invention.

### (1) First Part:

The first part in the present invention is a part (composition, particularly solid composition, e.g. a solid pharmaceutical composition for oral administration) comprising pioglitazone or a pharmaceutically acceptable salt thereof (particularly pioglitazone hydrochloride) and one or more excipients.

Excipients of the first part may comprise one or more diluents.

Furthermore, excipients of the first part may comprise one or more diluents and one or more binders.

Furthermore, excipients of the first part may comprise one or more diluents, one or more binders and one or more disintegrants.

Furthermore, excipients of the first part may comprise one or more diluents, one or more binders, one or more disintegrants and one or more lubricants.

Furthermore, excipients of the first part may comprise one or more diluents, one or more binders, one or more disintegrants, one or more lubricants and optional further excipient(s).

Excipients of the first part may be particularly selected from the group consisting of one or more diluents, one or more binders, one or more disintegrants, and one or more lubricants.

Examples of diluents of the first part include, without being limited to, mannitol, microcrystalline cellulose and/or pregelatinized starch. Of these, a particular diluent is mannitol.

Examples of binders of the first part include, without being limited to, copovidone, hydroxypropyl methylcellulose, hydroxypropyl cellulose and/or maize starch. Of these, copovidone is preferred.

Examples of disintegrants of the first part include, without being limited to, crospovidone, croscarmellose sodium, microcrystalline cellulose, pregelatinized starch and/or sodium starch glycolate. Of these, crospovidone is preferred.

Examples of lubricants of the first part include, without being limited to, sodium stearyl fumarate and/or magnesium stearate. Of these, sodium stearyl fumarate is preferred

It has surprisingly been observed that the use of sodium stearyl fumarate as lubricant in the first part results in a faster and more reproducible dissolution rate compared to tablets manufactured with magnesium stearate.

In more detail, the first part comprises usually one or more diluents (e.g. microcrystalline cellulose, pregelatinized starch and/or, particularly, mannitol), a binder (e.g. copovidone), a disintegrant (e.g. crospovidone), and a lubricant (e.g. sodium stearyl fumarate).

Suitably the pharmaceutical excipients used within the first part of the composition of this invention are conventional materials, such as e.g. mannitol (e.g. D-mannitol) as first diluent, microcrystalline cellulose or pregelatinized starch as second diluent, copovidone as binder, crospovidone as disintegrant, and/or sodium stearyl fumarate as lubricant,

The first part in the present invention may comprise pioglitazone hydrochloride, a first diluent and a second diluent.

Furthermore, the first part in the present invention may comprise pioglitazone hydrochloride, a first diluent, a second diluent and a binder.

Furthermore, the first part in the present invention may comprise pioglitazone hydrochloride, a first diluent, a second diluent, a binder and a disintegrant.

Furthermore, the first part in the present invention may comprise pioglitazone hydrochloride, a first diluent, a second diluent, a binder, a disintegrant and a lubricant.

Furthermore, the first part in the present invention may comprise pioglitazone hydrochloride, a first diluent, a second diluent, a binder, a disintegrant, a lubricant and optional one or more further ingredients.

For example, the first part in the present invention comprises pioglitazone hydrochloride, a first diluent, a second diluent, a binder, a disintegrant and a lubricant.

Preferably, the first part in the present invention is a part (composition) comprising or made of pioglitazone hydrochloride, one first diluent, one second diluent, one binder, one disintegrant and one lubricant.

The above-mentioned excipients of the first part (composition) typically comprises mannitol (e.g. D-mannitol) as a diluent or filler.

Further, the above-mentioned excipients of the first part (composition) typically comprises mannitol (e.g. D-mannitol) as first diluent.

Further, the above-mentioned excipients of the first part (composition) typically comprises the first diluent mannitol and one second diluent (e.g. microcrystalline cellulose or pregelatinized starch).

Further, the above-mentioned excipients of the first part (composition) typically comprises copovidone (also known as copolyvidone or Kollidon VA64) as binder.

Further, the above-mentioned excipients of the first part (composition) typically comprises crospovidone (also known as Kollidon CL-SF) as disintegrant.

Further, the above-mentioned excipients of the first part (composition) typically comprises sodium stearyl fumarate as lubricant or anti-adhesive.

A typical first part (composition) in the present invention contains or is made of pioglitazone hydrochloride, the first diluent mannitol, the second diluent microcrystalline cellulose or pregelatinized starch, the binder copovidone, the disintegrant crospovidone, and the lubricant sodium stearyl fumarate.

In one embodiment [embodiment A], the first part (composition) in the present invention comprises pioglitazone hydrochloride, the first diluent mannitol, the second diluent microcrystalline cellulose, the binder copovidone, the disintegrant crospovidone, and the lubricant sodium stearyl fumarate.

In another embodiment [embodiment B], the first part (composition) in the present invention comprises pioglitazone hydrochloride, the first diluent mannitol, the second diluent pregelatinized starch, the binder copovidone, the disintegrant crospovidone, and the lubricant sodium stearyl fumarate.

Among before-mentioned embodiments A and B, embodiment A is preferred.

Accordingly, in one embodiment of the present invention, the first part (composition) in the present invention comprises pioglitazone hydrochloride, a first diluent which is mannitol, a second diluent which is microcrystalline cellulose, a binder which is copovidone, a disintegrant which is crospovidone, and a lubricant which is sodium stearyl fumarate.

In another embodiment of the present invention, the first part (composition) in the present invention consists essentially of: pioglitazone hydrochloride, a first diluent which is mannitol, a second diluent which is microcrystalline cellulose, a binder which is copovidone, a disintegrant which is crospovidone, and a lubricant which is sodium stearyl fumarate.

In another embodiment of the present invention, the first part (composition) in the present invention consists essentially of: pioglitazone hydrochloride, a first diluent which is mannitol, a second diluent which is pregelatinized starch, a binder which is copovidone, a disintegrant which is crospovidone, and a lubricant which is sodium stearyl fumarate.

The content of the pioglitazone or a pharmaceutically acceptable salt thereof (particularly pioglitazone hydrochloride) may be 0.1-60 parts by weight, or 1-50 parts by weight, preferably 2-40 parts by weight, more preferably 5-30 parts by weight or, even more preferably, 5-20 parts by weight, relative to 100 parts by weight of the above-mentioned first part.

The compositions of this invention may contain the active ingredient pioglitazone or a pharmaceutically acceptable salt thereof (particularly pioglitazone hydrochloride) in the dosage range 1-100 mg, or 7.5-60 mg, or 15-60 mg, or 7.5-45 mg, each calculated for the active moiety pioglitazone (free form). Preferred dosages of pioglitazone are 15 mg, 30 mg and 45 mg of pioglitazone (corresponding to 16.53 mg, 33.06 mg and, respectively, 49.59 mg of pioglitazone hydrochloride). Preferably, the equivalent amount of pioglitazone hydrochloride to the pioglitazone free form is used in the compositions, namely, 16.53 mg, 33.06 mg and, respectively, 49.59 mg of pioglitazone hydrochloride.

The content of the first diluent (particularly mannitol) may be 5-99 parts by weight, or 10-95 parts by weight, preferably 20-90 parts by weight, more preferably 40-80 parts by weight or, even more preferably, 50-70 parts by weight, relative to 100 parts by weight of the above-mentioned first part.

The content of the second diluent (e.g. microcrystalline cellulose or pregelatinized starch) may be 1-70 parts by weight, or 1-50 parts by weight, preferably 5-40 parts by weight, more preferably 10-30 parts by weight or, even more preferably, 20-25 parts by weight, relative to 100 parts by weight of the above-mentioned first part.

The content of the binder (e.g. copovidone) may be 0.1-30 parts by weight, or 0.5-20 parts by weight, preferably 1-10 parts by weight, more preferably 1-5 parts by weight or, even more preferably, 1-3 parts by weight, relative to 100 parts by weight of the above-mentioned first part.

The content of the disintegrant (e.g. crospovidone) may be 0.1-30 parts by weight, or 0.5-20 parts by weight, preferably 1-10 parts by weight, more preferably 1-5 parts by weight or, even more preferably, 1-3 parts by weight, relative to 100 parts by weight of the above-mentioned first part.

The content of the lubricant (e.g. sodium stearyl fumarate) may be 0.5-20 parts by weight, or 0.1-10 parts by weight, preferably 0.1-4 parts by weight, more preferably 0.5-3 parts by weight or, even more preferably, 1-3 parts by weight, relative to 100 parts by weight of the above-mentioned first part.

In a further embodiment, the amount of sodium stearyl fumarate is preferably ≧1% by weight of the above-mentioned first part, e.g. 1-3% or 1-2%, more preferably ≧1.2%, e.g from 1.2% to 2%, most preferably about 2%, by weight of the above-mentioned first part.

The weight ratio of the pioglitazone or a pharmaceutically acceptable salt thereof (particularly pioglitazone hydrochloride) relative to the first diluent (particularly mannitol) may be (pioglitazone or a salt thereof: first diluent) 0.001-30:1, preferably 0.005-10:1, more preferably 0.01-1:1; or, even more preferably, 0.1-0.5:1 of pioglitazone hydrochloride:mannitol (e.g. about 0.14-0.15:1 or about 0.33:1).

The weight ratio of the pioglitazone or a pharmaceutically acceptable salt thereof (particularly pioglitazone hydrochloride) relative to the first and second diluent (particularly mannitol and either microcrystalline cellulose or pregelatinized starch) may be 0.001-30:1 (pioglitazone or a salt thereof: first and second diluent), preferably 0.005-10:1, more preferably 0.01-1:1; or, even more preferably, 0.05-0.5:1 of pioglitazone hydrochloride:sum of mannitol and either microcrystalline cellulose or pregelatinized starch (e.g. about 0.11:1 or about 0.24:1).

The weight ratio of the first diluent (particularly mannitol) relative to the second diluent (particularly either microcrystalline cellulose or pregelatinized starch) may be preferably from 2.22:1 to 4.33:1 (first diluent:second diluent), more preferably about 2.78:1 or about 3.24:1.

The first part (composition) according to this invention may comprise one or more of the following:

| | |
|---|---|
| 2-40% | pioglitazone (particularly pioglitazone hydrochloride), |
| 40-90% | one or more diluents, |
| 0.5-20% | one or more binders, |
| 0.5-20% | one or more disintegrants, and |
| 0.1-4% | one or more lubricants, |

wherein the percentages are by weight of the total first part.

The following ranges are preferred:

| | |
|---|---|
| 5-30% | pioglitazone (particularly pioglitazone hydrochloride), |
| 40-80% | diluent 1, |
| 5-40% | diluent 2, |
| 1-10% | binder, |
| 1-10% | disintegrant, |
| 0.5-3% | lubricant, |

wherein the percentages are by weight of the total first part.

The following ranges are more preferred:

| | |
|---|---|
| 5-20% | pioglitazone (particularly pioglitazone hydrochloride), |
| 50-70% | diluent 1, |
| 10-30% | diluent 2, |
| 1-3% | binder, |
| 1-3% | disintegrant, |
| 1-3% | lubricant, |

wherein the percentages are by weight of the total first part.

In a particular embodiment, the first part (composition) may comprise:

an intragranular portion containing pioglitazone hydrochloride, a first diluent (particularly mannitol), partly a second diluent (particularly microcrystalline cellulose), and a binder (particularly copovidone); and
an extragranular portion containing a disintegrant (particularly crospovidone), a lubricant (particularly sodium stearyl fumarate), and partly a second diluent (particularly microcrystalline cellulose).

In another embodiment of the present invention, the first part (composition) in the present invention consists essentially of:

an intragranular portion containing pioglitazone hydrochloride, a first diluent which is mannitol, a part of a second diluent which is microcrystalline cellulose, and a binder which is copovidone; and
an extragranular portion containing a disintegrant which is crospovidone, the remaining part of the second diluent which is microcrystalline cellulose, and a lubricant which is sodium stearyl fumarate.

To prepare the pioglitazone-containing first part (composition) of this invention, a granulate can be prepared, e.g., by a wet granulation process. Alternative methods for granulation of the active ingredient and excipients with a granulation liquid are fluid bed granulation or one pot granulation.

In the wet granulation process the granulation liquid is a solvent such as water, ethanol, methanol, isopropanol, acetone, or a mixture thereof, preferably purified water, and contains a binder such as copovidone. The solvent is a volatile component, which does not remain in the final product. The active ingredient pioglitazone HCl and the other excipients (e.g. mannitol and microcrystalline cellulose) with exception of the lubricant (e.g. sodium stearyl fumarate) and of the disintegrant (e.g. crospovidone) are premixed and granulated with the aqueous granulation liquid, e.g. using a high shear granulator. The wet granulation step is followed by an optional wet sieving step, drying and dry sieving of the granules. For example a fluid bed dryer can then be used for drying. The dried granules are sieved through an appropriate sieve to give the pioglitazone granulate. After dry sieving the granulate is optionally blended in a suitable blender. The lubricant (e.g. sodium stearyl fumarate) and the disintegrant (e.g. crospovidone) are blended in a suitable conventional blender such as a free fall blender to give a pre-mix, the pre-mix is sieved, and final mixed with the pioglitazone granulate in a suitable conventional blender such as a free fall blender to give the pioglitazone final blend.

Alternatively, but less preferred, in the wet granulation process the granulation liquid is a solvent such as water, ethanol, methanol, isopropanol, acetone, or a mixture thereof, preferably purified water, and contains a binder, such as copovidone, and a part of the second diluent (e.g. microcrystalline cellulose). The solvent is a volatile component, which does not remain in the final product. The active ingredient pioglitazone HCl and the other excipients (e.g. mannitol, remaining part of microcrystalline cellulose) with exception of the lubricant (e.g. sodium stearyl fumarate) and of the disintegrant (e.g. crospovidone) are premixed and granulated with the aqueous granulation liquid, e.g. using a high shear granulator. The wet granulation step is followed by an optional wet sieving step, drying and dry sieving of the granules. For example a fluid bed dryer can then be used for drying. The dried granules are sieved through an appropriate sieve to give the pioglitazone granulate. After dry sieving the granulate is optionally blended in a suitable blender. The lubricant (e.g. sodium stearyl fumarate) and the disintegrant (e.g. crospovidone) are blended in a suitable conventional blender such as a free fall blender to give a pre-mix, the pre-mix is sieved, and final mixed with the pioglitazone granulate in a suitable conventional blender such as a free fall blender to give the pioglitazone final blend.

In an embodiment, the second diluent (e.g. microcrystalline cellulose) may be optionally used intragranular, extragranular, or as a combination of both.

In a particular embodiment, one part of the second diluent (e.g. microcrystalline cellulose) may be present in the pioglitazone granulate and the remaining part thereof may be present in the extragranular portion of the pioglitazone final blend. For example, part of the second diluent (e.g. microcrystalline cellulose) may be added extragranular prior to final blending.

The amount of second diluent (e.g. microcrystalline cellulose) being present in the intragranular portion of the first part may be from 0 to 100%, preferably from 10 to 80%, more preferably from 20 to 50%, most preferably from 30 to 40% (e.g. about 34%), of total second diluent amount in the first part.

The amount of second diluent (e.g. microcrystalline cellulose) being present in the extragranular portion of the first part may be from 0 to 100%, preferably from 20 to 90%, more preferably from 50 to 80%, most preferably from 60 to 70% (e.g. about 66%), of total second diluent amount in the first part.

In an embodiment, the ratio of the intragranular second diluent (e.g. one part of microcrystalline cellulose) to the extragranular second diluent (e.g. remaining part of microcrystalline cellulose) may be from about 1:9 to about 9:1, or from about 1:4 to about 1:1, preferably from about 1:3 to about 1:1, more preferably from about 1:2.5 to about 1.15, even more preferably from about 3:7 to about 4:6, most preferably about 1:2.

Preferably, the pioglitazone final blend is prepared as follows: The wet granulation process the granulation liquid is a solvent such as water, ethanol, methanol, isopropanol, acetone, or a mixture thereof, preferably purified water, and contains a binder, such as copovidone. The solvent is a volatile component, which does not remain in the final product. The active ingredient pioglitazone HCl and the other excipients (e.g. mannitol, a part of the second diluent (e.g. microcrystalline cellulose, such as e.g. from about 20% to 50%, preferably from 30% to 40%, more preferably about one-third of total microcrystalline cellulose of the first part) with exception of the lubricant (e.g. sodium stearyl fumarate) and of the disintegrant (e.g. crospovidone) are premixed and granulated with the aqueous granulation liquid, e.g. using a high shear granulator. The wet granulation step is followed by an optional wet sieving step, drying and dry sieving of the granules. For example a fluid bed dryer can then be used for drying. The dried granules are sieved through an appropriate sieve to give the pioglitazone granulate. After dry sieving the granulate is optionally blended in a suitable blender. The remaining part of the second diluent (e.g. microcrystalline cellulose, pre-screened or unscreened, such as e.g. from about 50% to 80%, preferably from 60% to 70%, more preferably about two-third of total microcrystalline cellulose of the first part), the lubricant (e.g. sodium stearyl fumarate, pre-screened or unscreened) and the disintegrant (e.g. crospovidone, pre-screened or unscreened) are combined with the pioglitazone granulate (screened and optionally blended) for blending (e.g. in a suitable conventional blender such as a free fall blender). The blend is screened to give the pioglitazone final blend.

### (2) Second Part:

The second part in the present invention is a part (composition, particularly solid composition, e.g. a solid pharmaceutical composition for oral administration) comprising linagliptin or a pharmaceutically acceptable salt thereof (particularly linagliptin) and one or more excipients.

Excipients of the second part may comprise one or more diluents.

Furthermore, excipients of the second part may comprise one or more diluents and one or more binders.

Furthermore, excipients of the second part may comprise one or more diluents, one or more binders and one or more disintegrants.

Furthermore, excipients of the second part may comprise one or more diluents, one or more binders, one or more disintegrants and one or more lubricants.

Furthermore, excipients of the second part may comprise one or more diluents, one or more binders, one or more disintegrants, one or more lubricants and optional further excipient(s).

Excipients of the second part may be particularly selected from the group consisting of one or more diluents, one or more binders, one or more disintegrants, and one or more lubricants.

Examples of diluents of the second part include, without being limited to, cellulose powder, dibasic calciumphosphate (in particular anhydrous or dibasic calciumphosphate dihydrate), erythritol, low-substituted hydroxypropyl cellulose, mannitol, starch, pregelatinized starch and xylitol. The diluents pre-gelatinized starch and low-substituted hydroxypropyl cellulose show additional binder properties. Among these diluents mannitol and/or pregelatinized starch are preferred.

In case the second part (composition) according to the invention comprises one diluent, then the diluent is preferably mannitol or pregelatinized starch, more preferably mannitol.

Preferably, in case the second part (composition) according to the invention comprises two or more diluents, then the first diluent is preferably mannitol and the second diluent is selected from the group of diluents as described hereinbefore, more preferably pregelatinized starch, which shows additional binder properties.

Examples of binders of the second part include, without being limited to, copovidone, hydroxypropyl methylcellulose (HPMC), hydroxypropyl cellulose (HPC), polyvinylpyrrolidone (povidone), pregelatinized starch and low-substituted hydroxypropyl cellulose (L-HPC). Of these, copovidone and/or pregelatinized starch are preferred.

The above mentioned binders pregelatinized starch and L-HPC show additional diluent and disintegrant properties and can also be used as the second diluent or the disintegrant.

Examples of disintegrants of the second part include, without being limited to, crospovidone, low-substituted hydroxypropyl cellulose (L-HPC) and starches, such as native starches, in particular corn starch, and pregelatinized starch. Of these, corn starch is preferred.

Examples of lubricants of the second part include, without being limited to, talc, polyethylene glycol (particularly polyethylene glycol with a molecular weight in a range from about 4400 to about 9000), hydrogenated castor oil, fatty acid and salts of fatty acids, particularly the calcium, magnesium, sodium or potassium salts thereof, for example calcium behenate, calcium stearate, sodium stearyl fumarate or magnesium stearate. Of these, magnesium stearate is preferred.

In more detail, the second part comprises usually one or more diluents (e.g. mannitol and/or pregelatinized starch), a binder (e.g. copovidone), a disintegrant (e.g. corn starch), and a lubricant (e.g. magnesium stearate).

Suitably the pharmaceutical excipients used within the second part of the composition of this invention are conventional materials, such as e.g. mannitol (e.g. D-mannitol) as first diluent, pregelatinized starch as second diluent, copovidone as binder, corn starch as disintegrant, and/or magnesium stearate as lubricant.

The second part in the present invention may comprise linagliptin, a first diluent and a second diluent.

Furthermore, the second part in the present invention may comprise linagliptin, a first diluent, a second diluent and a binder.

Furthermore, the second part in the present invention may comprise linagliptin, a second diluent, a second diluent, a binder and a disintegrant.

Furthermore, the second part in the present invention may comprise linagliptin, a second diluent, a second diluent, a binder, a disintegrant and a lubricant.

Furthermore, the second part in the present invention may comprise linagliptin, a first diluent, a second diluent, a binder, a disintegrant, a lubricant and optional one or more further ingredients.

For example, the second part in the present invention comprises linagliptin, a first diluent, a second diluent, a binder, a disintegrant and a lubricant.

Preferably, the second part in the present invention is a part (composition) comprising or made of linagliptin, one first diluent, one second diluent, one binder, one disintegrant and one lubricant.

The above-mentioned excipients of the second part (composition) typically comprises mannitol (e.g. D-mannitol) as a diluent or filler.

Further, the above-mentioned excipients of the second part (composition) typically comprises mannitol (e.g. D-mannitol) as first diluent.

Further, the above-mentioned excipients of the second part (composition) typically comprises the first diluent mannitol and one second diluent (e.g. pregelatinized starch).

Further, the above-mentioned excipients of the second part (composition) typically comprises copovidone (also known as copolyvidone or Kollidon VA64) as binder.

Further, the above-mentioned excipients of the second part (composition) typically comprises corn starch (e.g. maize starch) as disintegrant.

Further, the above-mentioned excipients of the second part (composition) typically comprises magnesium stearate as lubricant or anti-adhesive.

A typical second part (composition) in the present invention contains or is made of linagliptin, the first diluent mannitol, the second diluent pregelatinized starch, the binder copovidone, the disintegrant corn starch, and the lubricant magnesium stearate.

Accordingly, in one embodiment of the present invention, the second part comprises linagliptin, a first diluent which is mannitol, a second diluent which is pregelatinized starch, a binder which is copovidone, a disintegrant which is corn starch, and a lubricant which is magnesium stearate.

In another embodiment of the present invention, the second part consists essentially of: linagliptin, a first diluent which is mannitol, a second diluent which is pregelatinized starch, a binder which is copovidone, a disintegrant which is corn starch, and a lubricant which is magnesium stearate.

The compositions of this invention may contain the active ingredient linagliptin in the dosage range 0.1-100 mg. Particular oral dosage strengths of linagliptin are 0.5 mg, 1 mg, 2.5 mg, 5 mg and 10 mg. More particular oral dosage strengths of linagliptin within this invention are 2.5 mg and 5 mg. A preferred oral dosage strength of linagliptin is 5 mg.

The second part (composition) according to this invention may comprise one or more of the following:

| | |
|---|---|
| 0.5-20% | active pharmaceutical ingredient (particularly linagliptin), |
| 40-90% | one or more diluents, |
| 0.5-20% | one or more binders, |
| 0.5-20% | one or more disintegrants, and |
| 0.1-4% | one or more lubricants, |

wherein the percentages are by weight of the total second part.

The following ranges are preferred:

| | |
|---|---|
| 0.5-10% | active pharmaceutical ingredient (particularly linagliptin), |
| 50-75% | diluent 1, |
| 0-15% | diluent 2, |
| 1-15% | binder, |
| 1-15% | disintegrant, |
| 0.5-3% | lubricant, |

wherein the percentages are by weight of the total second part.

The following ranges are more preferred:

| | |
|---|---|
| 0.5-7% | active pharmaceutical ingredient (particularly linagliptin), |
| 50-75% | diluent 1, |
| 5-15% | diluent 2, |
| 2-4% | binder, |
| 8-12% | disintegrant, |
| 0.5-2% | lubricant, |

wherein the percentages are by weight of the total second part.

To prepare the linagliptin-containing second part (composition) of this invention, a granulate can be prepared, e.g., by a wet granulation process. Alternative methods for granulation of the active ingredient and excipients with a granulation liquid are fluid bed granulation or one pot granulation.

In the wet granulation process the granulation liquid is a solvent such as water, ethanol, methanol, isopropanol, acetone, or a mixture thereof, preferably purified water, and contains a binder such as copovidone. The solvent is a volatile component, which does not remain in the final product. The active ingredient linagliptin and the other excipients (e.g. mannitol, pregelatinized starch and corn starch) with exception of the lubricant (e.g. magnesium stearate) are premixed and granulated with the aqueous granulation liquid, e.g. using a high shear granulator. The wet granulation step is followed by an optional wet sieving step, drying and dry sieving of the granules. For example a fluid bed dryer can then be used for drying. The dried granules are sieved through an appropriate sieve to give the linagliptin granulate. After dry sieving the granulate is optionally blended in a suitable blender. The lubricant (e.g. magnesium stearate) is final blended with the linagliptin granulate in a suitable conventional blender such as a free fall blender to give the linagliptin final blend.

For the preparation of tablets or tablet cores the final blend(s) are compressed into tablets. For the preparation of capsules the final blend(s) may be filled into a capsule.

Preferably, the pioglitazone final blend and the linagliptin final blend are compressed together into bilayer tablet cores, e.g. using a standard bilayer rotary tablet press.

Depending on the individual weight of each layer of the bilayer tablet core preferably the layer with the larger weight is chosen to be the first layer and the layer with the smaller weight to be the second layer. Less preferably the orientation of layers is the opposite. In case identical tablet layer weights of the first and second layer the more voluminous layer is preferably the first layer and only less preferably the second layer.

For the preparation of film-coated tablets a coating suspension is prepared and the compressed tablet cores are coated with the coating suspension to a weight gain of about 2-4%, preferably about 3%, using standard film coater (such as e.g. a perforated pan coater). The film coating solvent is a volatile component, which does not remain in the final product. A typical film-coat comprises a film coating agent, a plasticizer, a glidant, and optionally one or more pigments and colors. For example, the film coat may comprise hydroxypropylmethylcellulose (HPMC), propylene glycol, talc, titanium dioxide and, optionally, iron oxide (e.g. iron oxide yellow and/or red).

Alternatively, for preparing film-coated tablets of this invention the film coating suspension is prepared by using commercially available film coating pre-mixtures such as Opadry™ (which may be identical in qualitative and quantitative composition to using single film excipients). The single ingredients of the film-coat or the commercially available premixture such as Opadry™ is suspended or dissolved in the film coating solvent, preferably purified water at room temperature, for preparing the film-coating suspension.

To achieve most optimum physical and chemical stability the film-coating process is performed in such a way that the residual moisture of the final linagliptin/pioglitazone film-coated tablets is in the range of from 0.5 to 2.5%, preferably in the range of from 0.7 to 2.0%, more preferably in the range of from 0.8 to 1.5%, and most preferably in the range of from 0.9 to 1.4% by weight.

The term “linagliptin” as employed herein refers to linagliptin, a pharmaceutically acceptable salt thereof, a hydrate or solvate thereof, or a polymorphic form thereof. Crystalline forms are described in WO 2007/128721. Preferred crystalline forms are the polymorphs A and B described therein. In particular, linagliptin is the free base 1-[(4-methyl-quinazolin-2-yl)methyl]-3-methyl-7-(2-butyn-1-yl)-8-(3-(R)-amino-piperidin-1-yl)-xanthine. As linagliptin or a pharmaceutically acceptable salt thereof, linagliptin is preferred. Methods for the manufacture of linagliptin are described in the patent applications WO 2004/018468 and WO 2006/048427 for example.

Linagliptin is distinguished from structurally comparable DPP-4 inhibitors, as it combines exceptional potency and a long-lasting effect with favourable pharmacological properties, receptor selectivity and a favourable side-effect profile or bring about unexpected therapeutic advantages or improvements when used in combination with pioglitazone according to this invention.

The term “pioglitazone” as employed herein refers to pioglitazone, a pharmaceutically acceptable salt thereof, a hydrate or solvate thereof, or a polymorphic form thereof. Preferable examples of the salt of pioglitazone include salts with hydrochloric acid. As pioglitazone or a pharmaceutically acceptable salt thereof, pioglitazone hydrochloride is preferred. A preferred crystalline form of pioglitazone hydrochloride is the crystal form (polymorph) defined as Form I, e.g. in WO 03/026586.

In a preferred embodiment, the pharmaceutical compositions, dosage forms or tablets of this invention contain linagliptin in an amount of 5 mg, and pioglitazone in an amount of 15 mg, 30 mg or 45 mg.

In a further embodiment, the pharmaceutical compositions, dosage forms or tablets of this invention contain linagliptin in an amount of 2.5 mg, and pioglitazone in an amount of 15 mg, 30 mg or 45 mg.

In the pharmaceutical composition and pharmaceutical dosage form according to the invention the active pharmaceutical ingredients preferably may have a particle size distribution such that at least 90% of the respective active pharmaceutical ingredient particles, with regard to the distribution by volume, has a particle size smaller than 200 μm, i.e. X90<200 μm.

In particular for use in the pharmaceutical composition and pharmaceutical dosage form according to the invention linagliptin, for example a crystalline form thereof, preferably have a particle size distribution (by volume) such that at least 90% of the respective active pharmaceutical ingredient has a particle size smaller than 200 μm, i.e. X90<200 μm, more preferably X90≦150 μm. More preferably the particle size distribution is such that X90≦100 μm, even more preferably X90≦75 μm. In addition the particle size distribution is preferably such that X90>0.1 μm, more preferably X90≧1 μm, most preferably X90≧5 μm. Therefore preferred particle size distributions are such that 0.1 μm<X90<200 μm, particularly 0.1 μm<X90≦150 μm, more preferably 1 μm≦X90≦150 μm, even more preferably 5 μm≦X90≦100 μm. A preferred example of a particle size distribution of linagliptin is such that X90≦50 μm or 10 μm≦X90≦50 μm.

Furthermore for use in the pharmaceutical composition and pharmaceutical dosage form according to the invention linagliptin, for example a crystalline form thereof, preferably has a particle size distribution (by volume) such that X50≦90 μm, more preferably X50≦75 μm, even more preferably X50≦50 μm, most preferably X50≦40 μm. In addition the particle size distribution is preferably such that X50≦0.1 μm, more preferably X50≦0.5 μm, even more preferably X50≧4 μm. Therefore preferred particle size distributions are such that 0.1 μm≦X50≦90 μm, particularly 0.5 μm≦X50≦75 μm, more preferably 4 μm≦X50≦75 μm, even more preferably 4 μm≦X50≦50 μm. A preferred example is 8 μm≦X50≦40 μm.

Furthermore for use in the pharmaceutical composition and pharmaceutical dosage form according to the invention linagliptin, for example a crystalline form thereof, preferably has a particle size distribution (by volume) such that X10≧0.05 μm, more preferably X10≧0.1 μm, even more preferably X10≧0.5 μm.

For use in the pharmaceutical composition and pharmaceutical dosage form according to the invention pioglitazone (particularly pioglitazone hydrochloride), for example a crystalline form thereof, can be unmilled, milled (e.g. with a peg mill) or micronised. Milled pioglitazone hydrochloride may have, in one embodiment, a particle size distribution (by volume) such that at least 90% of the respective active pharmaceutical ingredient has a particle size smaller than 100 μm, i.e. X90<100 μm, and, optionally, X50 is 20-60 μm, and, further optionally, X10 is 5-10 μm. Unmilled pioglitazone hydrochloride may have a particle size distribution (by volume) such that at least 98% of the respective active pharmaceutical ingredient has a particle size smaller than 250 μm, i.e. X98<250 μm, and, optionally, X90<200 μm (e.g. 150-190 μm), and, further optionally, X50<100 μm (e.g. 70-90 μm), and, yet further optionally, X10 is 15-20 μm. In another embodiment, the median size of pioglitazone hydrochloride is preferably 1-50 μm, more preferably 2-30 μm, e.g. 2 to 25 μm or 2 to 15 μm (e.g. about 13 μm).

In a further embodiment, the following ranges of particle size distribution of pioglitazone HCl are more preferred:

Unmilled pioglitazone hydrochloride may have a particle size distribution (by volume) such that at least 98% of the respective active pharmaceutical ingredient has a particle size smaller than 450 μm, i.e. X98<450 μm, and, optionally, X90<300 μm (e.g. 1 μm<X90<300 μm), and, further optionally, X50<120 μm (e.g. 1 μm<X50<120 μm), and, yet further optionally, X10<50 μm (e.g. 0.1 μm<X10<50 μm, e.g. 15-20 μm).

Mannitol as mentioned hereinbefore and hereinafter is preferably D-mannitol (preferably of the beta-polymorphic form) and is preferably with a grade with small particle size suitable for (wet) granulation. Preferably, mannitol as mentioned hereinbefore and hereinafter is fine powdered. In the pioglitazone-containing first part (composition) of the invention, the mannitol may be crystalline powder (e.g. Pearlitol 25C™), milled (e.g. with a peg mill) or of directly compressible grade (e.g. Pearlitol SD200™). Mannitol of the first part may have a mean particle diameter of about 10 μm to about 180 μm, particularly of about 20 μm to about 40 μm.

Pregelatinized starch as mentioned hereinbefore and hereinafter is preferably a starch (e.g. maize (corn), potato or rice starch) that has been chemically and/or mechanically processed to rupture all or part of the starch granules. Particularly, partially pregelatinized starch has to be mentioned. An example is Starch 1500™ (Colorcon).

Copovidone as mentioned hereinbefore and hereinafter is preferably a copolymerisate of vinylpyrrolidon with vinyl acetate, preferably with a molecular weight from about 45000 to about 70000. An example is Polyvidon VA 64 or Kollidon™ VA 64 (BASF).

Crospovidone as mentioned hereinbefore and hereinafter is preferably a cross-linked and water insoluble form of PVP. An example is Kollidon™ CL-SF (BASF).

An example of sodium stearyl fumarate as mentioned hereinbefore and hereinafter is PRUV™.

Cellulose as mentioned hereinbefore and hereinafter is typically crystalline cellulose, preferably microcrystalline cellulose. An example is MCC 101.

Corn starch as mentioned hereinbefore and hereinafter is preferably a native starch. An example is Maize starch (extra white) (Roquette).

The pharmaceutical compositions (or formulations) may be packaged in a variety of ways. Generally, an article for distribution includes a container that contains the pharmaceutical composition in an appropriate form. Tablets are typically packed in an appropriate primary package for easy handling, distribution and storage and for assurance of proper stability of the composition at prolonged contact with the environment during storage. Primary containers for tablets may be bottles or blister packs.

A suitable bottle may be made from glass or polymer (preferably polypropylene (PP) or high density polyethylene (HD-PE)) and sealed with a screw cap. The screw cap may be provided with a child resistant safety closure (e.g. press-and-twist closure) for preventing or hampering access to the contents by children. If required (e.g. in regions with high humidity), by the additional use of a desiccant (such as e.g. bentonite clay, molecular sieves, or, preferably, silica gel) the shelf life of the packaged composition can be prolonged.

A suitable blister pack comprises or is formed of a top foil (which is breachable by the tablets) and a bottom part (which contains pockets for the tablets). The top foil may contain a metallic foil, particularly aluminum or aluminum alloy foil (e.g. having a thickness of 20 μm to 45 μm, preferably 20 μm to 25 μm) that is coated with a heat-sealing polymer layer on its inner side (sealing side). The bottom part may contain a multi-layer polymer foil (such as e.g. poly(vinyl chloride) (PVC) coated with poly(vinylidene chloride) (PVDC); or a PVC foil laminated with poly(chlorotrifluoroethylene) (PCTFE)) or a multi-layer polymer-metal-polymer foil (such as e.g. a cold-formable laminated PVC/aluminum/polyamide composition).

To ensure a long storage period especially under hot and wet climate conditions an additional overwrap or pouch made of a multi-layer polymer-metal-polymer foil (e.g. a laminated polyethylene/aluminum/polyester composition) may be used for the blister packs. Supplementary desiccant (such as e.g. bentonite clay, molecular sieves, or, preferably, silica gel) in this pouch package may prolong the shelf life even more under such harsh conditions.

The article may further comprise a label or package insert, which refer to instructions customarily included in commercial packages of therapeutic products, that may contain information about the indications, usage, dosage, administration, contraindications and/or warnings concerning the use of such therapeutic products. In one embodiment, the label or package inserts indicates that the composition can be used for any of the purposes described herein.

The pharmaceutical combinations of pioglitazone and linagliptin according to the present invention are suitable for use:

in treating and/or preventing (including slowing the progression and/or delaying the onset) of metabolic diseases, especially type 2 diabetes mellitus, obesity and conditions related thereto (e.g. diabetic complications),
either in type 2 diabetes patients who have not been previously treated with an antihyperglycemic agent,
or in type 2 diabetes patients with insufficient glycemic control despite therapy with one or two conventional antihyperglycemic agents selected from metformin, sulphonylureas, thiazolidinediones (e.g. pioglitazone), glinides, alpha-glucosidase blockers, GLP-1 or GLP-1 analogues, and insulin or insulin analogues;
optionally in combination with one or more other active substances.

In an embodiment, the present invention relates to the pharmaceutical compositions or combinations of pioglitazone and linagliptin according to this invention for use in treating and/or preventing (including slowing the progression and/or delaying the onset) of metabolic diseases, especially type 2 diabetes mellitus, obesity and conditions related thereto (e.g. diabetic complications), in type 2 diabetes patients with insufficient glycemic control despite therapy with pioglitazone alone.

In a further embodiment, the present invention relates to the pharmaceutical compositions or combinations of pioglitazone and linagliptin according to this invention for use in combination with metformin in treating and/or preventing (including slowing the progression and/or delaying the onset) of metabolic diseases, especially type 2 diabetes mellitus, obesity and conditions related thereto (e.g. diabetic complications), in type 2 diabetes patients with insufficient glycemic control despite dual combination therapy with pioglitazone and metformin.

In a further embodiment, the present invention relates to the pharmaceutical compositions or combinations of pioglitazone and linagliptin according to this invention for use in treating and/or preventing (including slowing the progression and/or delaying the onset) of metabolic diseases, especially type 2 diabetes mellitus, obesity and conditions related thereto (e.g. diabetic complications), in drug-naïve type 2 diabetes patients.

In a further embodiment, the present invention relates to the pharmaceutical compositions or combinations of pioglitazone and linagliptin according to this invention for use in treating and/or preventing (including slowing the progression and/or delaying the onset) of metabolic diseases, especially type 2 diabetes mellitus, obesity and conditions related thereto (e.g. diabetic complications), in type 2 diabetes patients for whom metformin therapy is inappropriate, e.g. due to intolerability or contraindication against metformin (e.g. patients at risk of gastrointestinal adverse events or of lactic acidose, such as e.g. renally impaired or elderly patients).

Further, the present invention also relates to the pharmaceutical compositions or combinations of pioglitazone and linagliptin according to this invention for use in one or more of the following methods of
preventing, slowing the progression of, delaying or treating a metabolic disorder or disease, such as e.g. type 1 diabetes mellitus, type 2 diabetes mellitus, impaired glucose tolerance (IGT), impaired fasting blood glucose (IFG), hyperglycemia, postprandial hyperglycemia, postabsorptive hyperglycemia, overweight, obesity, dyslipidemia, hyperlipidemia, hypercholesterolemia, hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, and/or metabolic syndrome;
improving and/or maintaining glycemic control and/or for reducing of fasting plasma glucose, of postprandial plasma glucose, of postabsorptive plasma glucose and/or of glycosylated hemoglobin HbA1c;
preventing, slowing, delaying or reversing progression from pre-diabetes, impaired glucose tolerance (IGT), impaired fasting blood glucose (IFG), insulin resistance and/or from metabolic syndrome to type 2 diabetes mellitus;
preventing, reducing the risk of, slowing the progression of, delaying or treating of complications of diabetes mellitus such as micro- and macrovascular diseases, such as nephropathy, micro- or macroalbuminuria, proteinuria, retinopathy, cataracts, neuropathy, learning or memory impairment, neurodegenerative or cognitive disorders, cardio- or cerebrovascular diseases, tissue ischaemia, diabetic foot or ulcus, atherosclerosis, hypertension, endothelial dysfunction, myocardial infarction, acute coronary syndrome, unstable angina pectoris, stable angina pectoris, peripheral arterial occlusive disease, cardiomyopathy, heart failure, heart rhythm disorders, vascular restenosis, and/or stroke;
reducing body weight and/or body fat or preventing an increase in body weight and/or body fat or facilitating a reduction in body weight and/or body fat;
preventing, slowing, delaying or treating the degeneration of pancreatic beta cells and/or the decline of the functionality of pancreatic beta cells and/or for improving, preserving and/or restoring the functionality of pancreatic beta cells and/or stimulating and/or restoring or protecting the functionality of pancreatic insulin secretion;
preventing, slowing, delaying or treating non alcoholic fatty liver disease (NAFLD) including hepatic steatosis, non-alcoholic steatohepatitis (NASH) and/or liver fibrosis (such as e.g. preventing, slowing the progression, delaying, attenuating, treating or reversing hepatic steatosis, (hepatic) inflammation and/or an abnormal accumulation of liver fat);
preventing, slowing the progression of, delaying or treating type 2 diabetes with failure to conventional antidiabetic mono- or combination therapy;
achieving a reduction in the dose of conventional antidiabetic medication required for adequate therapeutic effect;
reducing the risk for adverse effects associated with conventional antidiabetic medication (e.g. hypoglycemia); and/or
maintaining and/or improving the insulin sensitivity and/or for treating or preventing hyperinsulinemia and/or insulin resistance;
   in a patient in need thereof (such as, e.g., a patient as described herein, especially a type 2 diabetes patient), optionally in combination with one or more other therapeutic substances (such as e.g. selected from metformin, sulphonylureas, thiazolidinediones, glinides, alpha-glucosidase blockers, GLP-1 or GLP-1 analogues, and insulin or insulin analogues).

Further, the present invention also relates to the pharmaceutical compositions or combinations according to this invention for use in type 2 diabetes patients who are with diagnosed renal impairment (e.g. as diagnosed by impaired eGFR and/or impaired creatinine clearance, such as e.g. mild, moderate or severe renal impairment, or end stage renal disease) and/or who are at risk of developing renal complications, e.g. patients with or at risk of diabetic nephropathy (including e.g. chronic and progressive renal insufficiency, albuminuria and/or proteinuria).

The dose of linagliptin when administered orally is 0.5 mg to 10 mg per patient per day, preferably 2.5 mg to 10 mg or 1 mg to 5 mg per patient per day.

For example, the daily oral amount 5 mg linagliptin may be given in a once daily dosing regimen (i.e. 5 mg linagliptin once daily) or in a twice daily dosing regimen (i.e. 2.5 mg linagliptin twice daily).

Further, the present invention relates to a pharmaceutical composition according to this invention for use in a method of treating type 2 diabetes, said method comprising the oral administration of said composition containing effective amounts of the active ingredients (such as e.g. 5 mg/15 mg, 5 mg/30 mg or 5 mg/45 mg of linagliptin/pioglitazone) preferably once daily to the patient in need thereof.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

Further embodiments, features and advantages of the present invention may become apparent from the following examples. The following examples serve to illustrate, by way of example, the principles of the invention without restricting it.

### EXAMPLES

### 1a. Composition of BI 1356+Pioglitazone HCl FDC 5/15 mg Bilayer Tablet

| | | mg/Dosage |
|---|---|---|
| Material | rel. Mass % | unit |
| Pioglitazone HCl | 4.5917 | 16.530 |
| Mannitol | 32.4083 | 116.670 |
| Cellulose microcrystalline | 10.0000 | 36.000 |
| Copovidone | 1.0000 | 3.600 |
| Crospovidone | 1.0000 | 3.600 |
| Sodium stearyl fumarate | 1.0000 | 3.600 |
| Sum pioglitazone layer | | 180.000 |
| Linagliptin | 1.3889 | 5.000 |
| Mannitol | 36.3611 | 130.900 |
| Pregelatinized starch | 5.0000 | 18.000 |
| Maize starch | 5.0000 | 18.000 |
| Copovidone | 1.5000 | 5.400 |
| Magnesium stearate | 0.7500 | 2.700 |
| Sum linagliptin layer | | 180.000 |
| Sum tablet cores | 100.000 | 360.000 |
| Hydroxypropylmethylcellulose | 50.000 | 5.000 |
| Propylene glycol | 5.000 | 0.500 |
| Titan dioxide | 24.000 | 2.400 |
| Talc | 20.000 | 2.000 |
| Iron oxide, yellow | 1.000 | 0.100 |
| Iron oxide, red | — | — |
| Water, purified | | |
| Sum Coating | 100.000 | 10.000 |
| Sum Filmtablets | | 370.0 |

### 1b. Composition of BI 1356+Pioglitazone HCl FDC 5/30 mg Bilayer Tablet

| | | mg/Dosage |
|---|---|---|
| Material | rel. Mass % | unit |
| Pioglitazone HCl | 9.1833 | 33.0600 |
| Mannitol | 27.8167 | 100.1400 |
| Cellulose microcrystalline | 10.0000 | 36.0000 |
| Copovidone | 1.0000 | 3.6000 |
| Crospovidone | 1.5000 | 5.4000 |
| Sodium stearyl fumarate | 1.5000 | 5.4000 |
| Sum pioglitazone layer | | 180.0000 |
| Linagliptin | 1.3889 | 5.0000 |
| Mannitol | 36.3611 | 130.9000 |
| Pregelatinized starch | 5.0000 | 18.0000 |
| Maize starch | 5.0000 | 18.0000 |
| Copovidone | 1.5000 | 5.4000 |
| Magnesium stearate | 0.7500 | 2.7000 |
| Sum linagliptin layer | | 180.0000 |
| Sum tablet cores | 100.0000 | 360.0000 |
| Hydroxypropylmethylcellulose | 50.0000 | 5.0000 |
| Propylene glycol | 5.0000 | 0.5000 |
| Titan dioxide | 21.0000 | 2.1000 |
| Talc | 20.0000 | 2.0000 |
| Iron oxide, yellow | 3.7500 | 0.3750 |
| Iron oxide, red | 0.2500 | 0.0250 |
| Water, purified | | |
| Sum Coating | 100.0000 | 10.0000 |
| Sum Filmtablets | | 370.0000 |

### 1c. Composition of BI 1356+Pioglitazone HCl FDC 5/45 mg Bilayer Tablet

| | | mg/Dosage |
|---|---|---|
| Material | rel. Mass % | unit |
| Pioglitazone HCl | 11.0200 | 49.5900 |
| Mannitol | 33.3800 | 150.2100 |
| Cellulose microcrystalline | 12.0000 | 54.0000 |
| Copovidone | 1.2000 | 5.4000 |
| Crospovidone | 1.2000 | 5.4000 |
| Sodium stearyl fumarate | 1.2000 | 5.4000 |
| Sum pioglitazone layer | | 270.0000 |
| Linagliptin | 1.1111 | 5.0000 |
| Mannitol | 29.0889 | 130.9000 |
| Pregelatinized starch | 4.0000 | 18.0000 |
| Maize starch | 4.0000 | 18.0000 |
| Copovidone | 1.2000 | 5.4000 |
| Magnesium stearate | 0.6000 | 2.7000 |
| Sum linagliptin layer | | 180.0000 |
| Sum tablet cores | 100.0000 | 450.0000 |
| Hydroxypropylmethylcellulose | 50.0000 | 6.0000 |
| Propylene glycol | 5.0000 | 0.6000 |
| Titan dioxide | 21.0000 | 2.5200 |
| Talc | 20.0000 | 2.4000 |
| Iron oxide, yellow | 2.0000 | 0.2400 |
| Iron oxide, red | 2.0000 | 0.2400 |
| Water, purified | | |
| Sum Coating | 100.0000 | 12.0000 |
| Sum Filmtablets | | 462.0000 |

### 1a′. Alternative composition of BI 1356+Pioglitazone HCl FDC 5/15 mg Bilayer Tablet

| | mg/Dosage |
|---|---|
| | unit 5/15 mg |
| Material | tablet |
| Pioglitazone HCl | 16.53 |
| Mannitol | 50.07 |
| Cellulose microcrystalline | 18.00 |
| Copovidone | 1.80 |
| Crospovidone | 1.80 |
| Sodium stearyl fumarate | 1.80 |
| Sum pioglitazone layer | 90.00 |
| Linagliptin | 5.00 |
| Mannitol | 130.90 |
| Pregelatinized starch | 18.00 |
| Maize starch | 18.00 |
| Copovidone | 5.40 |
| Magnesium stearate | 2.70 |
| Sum linagliptin layer | 180.00 |
| Sum tablet cores | 270.00 |
| Hydroxypropylmethylcellulose | 4.00 |
| Propylene glycol | 0.40 |
| Titan dioxide | 1.99 |
| Talc | 1.60 |
| Iron oxide, yellow | 0.01 |
| Iron oxide, red | — |
| Water, purified | |
| Sum Coating | 8.00 |
| Sum Filmtablets | 278.0 |

### 1″. Alternative composition of BI 1356+Pioglitazone FDC Bilayer Tablets

### Qualitative and Quantitative Composition:

| | Dosage strength Linagliptin/Pioglitazone | | | | | |
|---|---|---|---|---|---|---|
| | 5 mg/15 mg | | 5 mg/30 mg | | 5 mg/45 mg | |
| | [mg per film- | | [mg per film- | | [mg per film- | |
| Ingredients | coated tablet] | (%) | coated tablet] | (%) | coated tablet] | (%) |
| Linagliptin | 5.0000 | 2.7778 | 5.0000 | 2.7778 | 5.0000 | 2.7778 |
| Mannitol | 130.9000 | 72.7222 | 130.9000 | 72.7222 | 130.9000 | 72.7222 |
| Starch, pregelatinized | 18.0000 | 10.0000 | 18.0000 | 10.0000 | 18.0000 | 10.0000 |
| Maize starch | 18.0000 | 10.0000 | 18.0000 | 10.0000 | 18.0000 | 10.0000 |
| Copovidone | 5.4000 | 3.0000 | 5.4000 | 3.0000 | 5.4000 | 3.0000 |
| Magnesium stearate | 2.7000 | 1.5000 | 2.7000 | 1.5000 | 2.7000 | 1.5000 |
| Subtotal | 180.0000 | 100.0000 | 180.0000 | 100.0000 | 180.0000 | 100.0000 |
| Linagliptin Layer | | | | | | |
| Pioglitazone | 16.5300 | 18.3667 | 33.0600 | 18.3667 | 49.5900 | 18.3667 |
| hydrochloride | | | | | | |
| Mannitol | 50.0700 | 55.6333 | 100.1400 | 55.6333 | 150.2100 | 55.6333 |
| Cellulose, | 18.0000 | 20.0000 | 36.0000 | 20.0000 | 54.0000 | 20.0000 |
| microcrystalline | | | | | | |
| Copovidone | 1.8000 | 2.0000 | 3.6000 | 2.0000 | 5.4000 | 2.0000 |
| Crospovidone | 1.8000 | 2.0000 | 3.6000 | 2.0000 | 5.4000 | 2.0000 |
| Sodium stearyl | 1.8000 | 2.0000 | 3.6000 | 2.0000 | 5.4000 | 2.0000 |
| fumarate | | | | | | |
| Subtotal | 90.0000 | 100.0000 | 180.0000 | 100.0000 | 270.0000 | 100.0000 |
| Pioglitazone Layer | | | | | | |
| Opadry yellow | 8.0000 | 100.0000 | — | — | — | — |
| Opadry orange | — | — | 10.0000 | 100.0000 | — | — |
| Opadry pink | — | — | — | — | 12.0000 | 100.0000 |
| Subtotal Filmcoat | 8.0000 | 100.0000 | 10.0000 | 100.0000 | 12.0000 | 100.0000 |
| Total weight | 278.0000 | 100.0000 | 370.0000 | 100.0000 | 462.0000 | 100.0000 |
| Film-coated Tablet | | | | | | |

Qualitative and quantitative composition of Opadry® yellow, Opadry® orange and Opadry® pink film coat for linagliptin/pioglitazone film-coated tablets:

| | Quantity | | | |
|---|---|---|---|---|
| | [% w/w] | | | |
| | Opadry ® | Opadry ® | Opadry ® | |
| Ingredient | yellow | orange | pink | Function |
| Hypromellose 2910 | 50.0000 | 50.0000 | 50.0000 | Film-forming |
| | | | | agent |
| Titanium dioxide | 24.8500 | 21.0000 | 21.0000 | Pigment |
| Talc | 20.0000 | 20.0000 | 20.0000 | Anti-adherent |
| Propylene glycol | 5.0000 | 5.0000 | 5.0000 | Plasticizer |
| Iron oxide, yellow | 0.1500 | 3.7500 | 2.0000 | Pigment |
| Iron oxide, red | — | 0.2500 | 2.0000 | Pigment |
| Total | 100.0000 | 100.0000 | 100.0000 | — |

Manufacturing process of the exemplary compositions:

a) Linagliptin final blend:
i.) Granulation liquid for linagliptin final blend (step 1):
Copovidone is dispensed in water, purified.
ii.) Granulate for linagliptin final blend (step 2):

Mannitol, starch, pregelatinized and maize starch are screened through a suitable screen and pre-mixed in an appropriate high-shear mixer altogether with linagliptin, which has been optionally screened through a suitable screen.

Alternatively, mannitol, pregelatinized starch, maize starch and linagliptin are screened through a suitable screen and pre-mixed in an appropriate high-shear mixer.

Preferably the screening machine is directly linked to the granulator and the materials are directly screened into the granulator. Optionally the material transfer into the granulator and the screening step are combined using vacuum transfer via the screen into the granulator. In any case linagliptin is preferably screened in between the other excipients, less preferably before or after the excipients.

The pre-mix is moistened with the granulation liquid and granulated using an appropriate high-shear mixer. The wet granulate is optionally wet screened through a suitable screen. Subsequently, the wet granulate is dried in a fluid bed dryer and consecutively screened through a suitable screen; optionally, the screened granulate may be blended in an appropriate free-fall blender.

Alternatively a screening step takes place during drying, for which the drying process is paused and continued after screening. Subsequently, the dried granulate is optionally screened a second time followed by an optional blending step in an appropriate blender such as a free fall blender.

### iii.) Linagliptin Final Blend (Step 3):

For the linagliptin final blend optionally either fractions or multiples of entire linagliptin granulate batches are combined without impact on the quality and manufacturability of the drug product.

Pre-screened magnesium stearate is added to the screened and optionally blended granulate and subsequently final blending is performed in an appropriate free-fall blender.

### b) Pioglitazone Final Blend:

### i.) Granulation Liquid for Pioglitazone Final Blend (Step 4):

Copovidone is dispensed in water, purified.

### ii.) Granulate for Pioglitazone Final Blend (Step 5):

Pioglitazone hydrochloride, mannitol and cellulose (microcrystalline; e.g. a part or entire amount thereof) are screened through a suitable screen and pre-mixed in an appropriate high-shear mixer. The pre-mix is moistened with the granulation liquid and granulated using an appropriate high-shear mixer. The wet granulate is optionally wet screened through a suitable screen. Subsequently, the wet granulate is dried in a fluid bed dryer and consecutively screened through a suitable screen;

subsequently, the screened granulate may be blended in an appropriate free-fall blender.

### iii.) Pioglitazone Final Blend (Step 6):

For the pioglitazone final blend optionally either fractions or multiples of entire pioglitazone granulate batches are combined without impact on the quality and manufacturability of the drug product;

To obtain the pioglitazone final blend:

Variant 1: crospovidone and sodium stearyl fumarate are pre-blended and screened and consecutively combined with the screened pioglitazone granulate to perform the final blending; or
Variant 2: Cellulose (microcrystalline; e.g. remaining part thereof), crospovidone and sodium stearyl fumarate are pre-blended and screened and consecutively combined with the screened pioglitazone granulate to perform the final blending; or
Variant 3: Cellulose (microcrystalline; e.g. remaining part thereof), crospovidone and sodium stearyl fumarate are combined with the screened and optionally blended pioglitazone granulate to perform a blending step. Consecutively the blend is screened and final blending takes place.

All screening and pre-/final-blending process steps are performed using suitable screens and appropriate free-fall blenders, respectively;

Optionally, part of the microcrystalline cellulose (e.g. 30-40% of total amount thereof, such as e.g. about 34%) may present in the pioglitazone granulate and the remaining part thereof (e.g. 60-70% of total amount thereof, such as e.g. about 66%) may be present in the extragranular portion of the final blend. The ratio of the intragranular microcrystalline cellulose to the extragranular microcrystalline cellulose may be from about 1:4 to about 1:1, preferably from about 1:3 to about 1:1, more preferably from about 1:2.5 to about 1.15, even more preferably from about 3:7 to about 4:6, most preferably about 1:2.

### c) Linagliptin/Pioglitazone Bilayer Tablet Cores (Step 7):

The pioglitazone final blend and the linagliptin final blend are compressed into bilayer tablet cores using a standard bilayer rotary tablet press;

### d) Film-Coating Suspension (Step 8):

Hypromellose (HPMC), talc, propylene glycol, titanium dioxide, iron oxide yellow and/or, depending on the dosage strength, iron oxide red are dispersed in water, purified to achieve an aqueous film-coating suspension; or alternatively a commercially available premixture such as Opadry® of identical qualitative and quantitative composition is used instead of the single film ingredients. Depending on dosage strength Opadry® yellow, Opadry® orange or Opadry® pink is dispersed in purified water to obtain an aqueous film-coating suspension).

### e) Linagliptin/Pioglitazone Film-Coated Tablets (Step 9):

The linagliptin/pioglitazone bilayer tablet cores are coated with the film-coating suspension in a drum coater to produce linagliptin/pioglitazone film-coated tablets. Preferably a perforated drum coater is used.

1.′″ Formulation Variant with Extragranular Excipient in the Pioglitazone-Containing Part or Layer

### Qualitative and Quantitative Composition:

| | Dosage strength Pioglitazone | | | | | |
|---|---|---|---|---|---|---|
| | 15 mg | | 30 mg | | 45 mg | |
| | [mg per film- | | [mg per film- | | [mg per film- | |
| Ingredients | coated tablet] | (%) | coated tablet] | (%) | coated tablet] | (%) |
| Pioglitazone | 16.5300 | 18.3667 | 33.0600 | 18.3667 | 49.5900 | 18.3667 |
| hydrochloride | | | | | | |
| Mannitol | 50.0700 | 55.6333 | 100.1400 | 55.6333 | 150.2100 | 55.6333 |
| Cellulose, | 6.0000 | 6.6667 | 12.0000 | 6.6667 | 18.0000 | 6.6667 |
| microcrystalline | | | | | | |
| Copovidone | 1.8000 | 2.0000 | 3.6000 | 2.0000 | 5.4000 | 2.0000 |
| Subtotal granulate | 74.4000 | 82.6667 | 148.8000 | 82.6667 | 223.2000 | 82.6667 |
| Cellulose, | 12.0000 | 13.3333 | 24.0000 | 13.3333 | 36.0000 | 13.3333 |
| microcrystalline | | | | | | |
| Crospovidone | 1.8000 | 2.0000 | 3.6000 | 2.0000 | 5.4000 | 2.0000 |
| Sodium stearyl | 1.8000 | 2.0000 | 3.6000 | 2.0000 | 5.4000 | 2.0000 |
| fumarate | | | | | | |
| Total | 90.0000 | 100.0000 | 180.0000 | 100.0000 | 270.0000 | 100.0000 |
| Pioglitazone Part | | | | | | |

Copovidone is dissolved in purified water to produce a granulation liquid. Pioglitazone hydrochloride, mannitol and a part of microcrystalline cellulose are screened through a suitable screen and blended in a suitable mixer (e.g. high-shear mixer) to produce a pre-mix. The pre-mix is moistened with the granulation liquid and subsequently granulated (e.g. using a suitable high-shear mixer). The wet granulate is optionally wet sieved through a suitable sieve. Subsequently, the wet granulate is dried in a fluid bed dryer and consecutively screened through a suitable screen; subsequently, the screened granulate may be blended in an appropriate free-fall blender.

The remaining part of microcrystalline cellulose, crospovidone and sodium stearyl fumarate are added extragranular to the screened and optionally blended granulate to perform a blending step. Consecutively the blend is screened and final blending takes place in a suitable blender to produce the final blend.

2. Composition of BI 1356+Pioglitazone HCl FDC Bilayer Tablet (with the Variant Pregelatinized Starch as Second Diluent)

| | | | mg/ |
|---|---|---|---|
| | | | Dosage |
| | mg/Dosage | mg/Dosage | unit 5/ |
| Material | unit 5/15 mg | unit 5/30 mg | 45 mg |
| Pioglitazone HCl | 16.53 | 33.06 | 49.59 |
| Mannitol | 109.47 | 101.94 | 152.91 |
| Pregelatinized starch | 45.00 | 36.00 | 54.00 |
| Copovidone | 3.60 | 3.60 | 5.40 |
| Crospovidone | 1.80 | 1.80 | 2.70 |
| Sodium stearyl fumarate | 3.60 | 3.60 | 5.40 |
| Sum pioglitazone layer | 180.00 | 180.00 | 270.00 |
| Linagliptin | 5.00 | 5.00 | 5.00 |
| Mannitol | 130.90 | 130.90 | 130.90 |
| Pregelatinized starch | 18.00 | 18.00 | 18.00 |
| Maize starch | 18.00 | 18.00 | 18.00 |
| Copovidone | 5.40 | 5.40 | 5.40 |
| Magnesium stearate | 2.70 | 2.70 | 2.70 |
| Sum linagliptin layer | 180.00 | 180.00 | 180.00 |
| Sum tablet cores | 360.00 | 360.00 | 450.00 |
| Hydroxypropylmethylcellulose | 5.00 | 5.00 | 6.00 |
| Propylene glycol | 0.50 | 0.50 | 0.60 |
| Titan dioxide | 2.40 | 2.10 | 2.52 |
| Talc | 2.00 | 2.00 | 2.40 |
| Iron oxide, yellow | 0.10 | 0.375 | 0.24 |
| Iron oxide, red | — | 0.025 | 0.24 |
| Water, purified | | | |
| Sum Coating | 10.000 | 10.000 | 12.000 |
| Sum Filmtablets | 370.0 | 370.0 | 462.0 |

This composition and tablet is prepared by a similar or analogous process as that described herein for the variants with microcrystalline cellulose as second diluent.

### 3. Lubricant Selection for Optimization:

Sodium stearyl fumarate is preferred over magnesium stearate as lubricant, e.g. since it does not show some disadvantages found for magnesium stearate for over blending and/or reduction of dissolution of active ingredients (APIs). Rather sodium stearyl fumarate shows with sieving step and longer blending time of pioglitazone granulate and lubricant, increasing dissolution rate for pioglitazone.

For example, with the use of magnesium stearate (compared to the use of sodium stearyl fumarate) up to 25% less dissolution of pioglitazone after 5 minutes, 19% less dissolution of pioglitazone after 10 minutes, 15% less dissolution of pioglitazone at 15 minutes and/or not 100% receivable dissolution of pioglitazone at 45 minutes at pH2 with 50 UpM is found. Dissolution-Medium: pH 2.0: 0.01 M HCl/0.3 M KCl; Paddle, 900 mL, 50 rpm, 37.0° C.

For further example, with reduction of sodium stearyl fumarate from 2% by weight to e.g. 1 by weight of the pioglitazone layer, decreasing of dissolution of pioglitazone of 10-20 (and more) at pH 2 at 50 Upm in vitro is found. The amount of sodium stearyl fumarate is preferably ≧1% by weight of the pioglitazone layer, e.g. 1-3% or 1-2%, more preferably ≧1.2%, e.g., from 1.2% to 2%, most preferably about 2%, by weight of the pioglitazone layer.

### 4. Comparison of Bilayer Tablet and Monolayer Tablet in View of Stability Results:

### a) Composition of Monolayer Tablet:

| Material: | mg/Dosage unit |
|---|---|
| Linagliptin | 5 |
| Pioglitazone HCl, unmilled | 49.59 |
| Mannitol fine | 40 |
| Mannitol M 200 | 165.96 |
| Crospovidone | 5.4 |
| Magnesium stearate | 4.05 |

### b) Composition of Bilayer Tablet:

| | mg/Dosage unit |
|---|---|
| Material Layer 1: | |
| Pioglitazone HCl | 49.59 |
| Mannitol fine | 22.7 |
| Mannitol M 200 | 188.26 |
| Crospovidone | 5.4 |
| Magnesium stearate | 4.05 |
| Material Layer 2: | |
| Linagliptin | 5 |
| Mannitol fine | 62.95 |
| Pregelatinized starch | 9 |
| Maize starch undried | 9 |
| Copovidone | 2.7 |
| Magnesium stearate | 1.35 |

Stability results (40° C., 75% rh, open, after 4-6 week):

Version a) monolayer tablet (film coated, 5/45 mg):
degradation after 4 weeks: linagliptin about 11%, pioglitazone <0.2% Version b) bilayer tablet (film coated, 5/45 mg with roller compaction layer of pioglitazone):
degradation after 6 weeks: linagliptin <0.2%, pioglitazone <0.2%

### 5. Stability/Assay Results of Example 1c (5/45 mg Film Coated Tablet):

Assay results: linagliptin 102.1%, pioglitazone 99.2% at start
Dissolution results (Q at 15 min, pH 2.0): linagliptin 102%, pioglitazone 95%

Dissolution Profile Pioglitazone:

10 min: 92%, 15 min: 95%, 30 min: 97%, 45 min 97%

### Dissolution Profile Linagliptin:

10 min: 100%, 15 min: 102%, 30 min: 103%, 45 min 103%

Stability results (40° C., 75% rh, open, after 4 week):
degradation: linagliptin about 0.1%, pioglitazone <0.1%,
assay: linagliptin 101.2%, pioglitazone 99.5%

Stability results (40° C., 75% rh, open, after 9 week):
degradation: linagliptin about 0.4%, pioglitazone <0.1%,
assay: linagliptin 99.5%, pioglitazone 99.0%

What is claimed is:

1. A pharmaceutical composition comprising a first part and a second part, wherein:
the first part comprises pioglitazone, or the hydrochloride salt thereof, and one or more excipients, and
the second part comprises linagliptin and one or more excipients.

2. The composition of claim 1, wherein the first part comprises pioglitazone hydrochloride, a first diluent which is mannitol, a second diluent which is microcrystalline cellulose, a binder which is copovidone, a disintegrant which is crospovidone, and a lubricant which is sodium stearyl fumarate.

3. The composition of claim 1, wherein the second part comprises linagliptin, a first diluent which is mannitol, a second diluent which is pregelatinized starch, a binder which is copovidone, a disintegrant which is corn starch, and a lubricant which is magnesium stearate.

4. The composition of claim 1 in solid oral dosage form selected from a capsule, a tablet or a film-coated tablet.

5. The composition of claim 1 in the form of a bilayer tablet.

6. The composition of claim 5, which is a film-coated bilayer tablet.

7. The composition of claim 6, wherein the film-coat comprises hydroxypropylmethylcellulose (HPMC), polypropylene glycol, talc, titanium dioxide and an iron oxide.

8. The composition of claim 1, wherein pioglitazone, or the hydrochloride salt thereof, is present in an amount of 15 mg, 30 mg or 45 mg based on the weight of pioglitazone.

9. The composition of claim 1, wherein linagliptin is present in an amount of 5 mg.

10. The composition of claim 1, wherein linagliptin is present in an amount of 2.5 mg.

11. The composition of claim 1, wherein the first part comprises an intragranular portion containing pioglitazone hydrochloride, a first diluent which is mannitol, a partial amount of a second diluent which is microcrystalline cellulose, and a binder which is copovidone; and an extragranular portion containing a disintegrant which is crospovidone, a lubricant which is sodium stearyl fumarate, and the remaining partial amount of the second diluent which is microcrystalline cellulose.

12. The composition of claim 11, wherein the amount of the microcrystalline cellulose contained in the intragranular portion is from 10 to 80% of the total amount of microcrystalline cellulose comprised in the first part.

13. The composition of claim 11, wherein the amount of the microcrystalline cellulose contained in the extragranular portion is from 20 to 90% of the total amount of microcrystalline cellulose comprised in the first part.

14. The composition of claim 11, wherein from 30% to 40% of the total amount of microcrystalline cellulose is present intragranular, and wherein from 60% to 70% of the total amount of microcrystalline cellulose is present extragranular.

15. The composition of claim 11, wherein the ratio of intragranular microcrystalline cellulose to extragranular microcrystalline cellulose is about 1:2.

16. A process for the preparation of the first part of the pharmaceutical composition of claim 1, said process comprising
a. dissolving a binder in a solvent to produce a granulation liquid,
b. blending pioglitazone HCl, a first diluent, and a part of the second diluent to produce a pre-mix,
c. moistening and granulating the pre-mix with the granulation liquid,
d. optionally wet sieving, drying and dry sieving of the obtained pioglitazone-containing granulate, and
e. combining the pioglitazone-containing granulate with a lubricant, a disintegrant, and the remaining part of the second diluent for final blending.

17. A process for the preparation of the second part of the pharmaceutical composition of claim 1, said process comprising
a. dissolving a binder in a solvent to produce a granulation liquid,
b. blending linagliptin, a first diluent, a second diluent, and a disintegrant to produce a pre-mix,
c. moistening and granulating the pre-mix with the granulation liquid,
d. optionally wet sieving, drying and dry sieving of the obtained linagliptin-containing granulate, and
e. adding a lubricant to the linagliptin-containing granulate for final blending.

18. A process for the preparation of a pharmaceutical composition comprising (i) a first part comprising the hydrochloride salt of pioglitazone (pioglitazone HCl) and one or more excipients, and (ii) a second part comprising linagliptin and one or more excipients, said process comprising:
combining the pioglitazone HCl final blend obtained in step (e) of claim 16 and a linagliptin final blend, which linagliptin final blend is obtained by
f. dissolving a binder in a solvent to produce a granulation liquid,
g. blending linagliptin, a first diluent, a second diluent, and a disintegrant to produce a pre-mix,
h. moistening and granulating the pre-mix with the granulation liquid,
i. optionally wet sieving, drying and dry sieving of the obtained linagliptin-containing granulate, and
j. adding a lubricant to the linagliptin-containing granulate for final blending;
compressing the pioglitazone HCl final blend and the linagliptin final blend into a bilayer tablet core,
preparing a coating suspension, and
coating the tablet core with the coating suspension to produce a film-coated bilayer tablet.

19. A method of using the pharmaceutical composition of claim 1 for treating type 2 diabetes or obesity.

20. A method of treating type 2 diabetes, said method comprising the oral administration of the composition of claim 1 once or twice daily to the patient.
